(19) United States
(12) Patent Application Publication
(10) Pub. No.: US 2018/0133950 A1
(43) Pub. Date: May 17, 2018

(54) DEVICES AND METHODS FOR PRODUCTION OF HOLLOW CYLINDRICAL STRUCTURES AND HOLLOW CYLINDRICAL STRUCTURES

(71) Applicant: CASE WESTERN RESERVE UNIVERSITY, CLEVELAND, OH (US)

(72) Inventors: Umit Levent Erol, Cleveland, OH (US); Mark Weidenbecher, Shaker Heights, OH (US); Ozan Akkus, Cleveland Heights, OH (US); Mohammad Mofidfar, Atlanta, GA (US)

(21) Appl. No.: 15/814,997

(22) Filed: Nov. 16, 2017

Related U.S. Application Data
(60) Provisional application No. 62/432,933, filed on Dec. 12, 2016.
(60) Provisional application No. 62/423,408, filed on Nov. 17, 2016.

(51) Int. Cl. B29C 53/60; A61L 27/56; A61L 27/12

(52) U.S. Cl. CPC B29C 53/60; B29L 2031/7542; A61L 27/12; A61L 27/56

(57) ABSTRACT

A device suitable for constructing hollow cylindrical structures, particularly useful as medical devices. The devices are versatile in nature and can fabricate hollow cylindrical structures having single or multiple layers of one or more different materials, including biomaterials. Methods for utilizing the devices and fabricating hollow cylindrical structures are also disclosed. Fabricated hollow cylindrical structures are described herein. including embodiments incorporating a biomaterial that facilitates cell attachment and tissue integration.

### GOVERNMENT LICENSE RIGHTS

This invention was made with government support under Contract DMR-1306665 awarded by the National Science Foundation. The U.S. government has certain rights in the invention.

### FIELD OF THE INVENTION

The present invention relates to devices suitable for constructing hollow cylindrical structures, particularly useful as medical devices. The devices are versatile in nature and can fabricate hollow cylindrical structures having single or multiple layers of one or more different materials, including biomaterials. Methods for utilizing the devices and fabricating hollow cylindrical structures are also disclosed. Fabricated hollow cylindrical structures are described herein, including embodiments incorporating a biomaterial that facilitates cell attachment and tissue integration.

### BACKGROUND OF THE INVENTION

Tubular cylindrical medical devices are used in numerous medical applications in urological, cardiovascular, orthopaedic, throat surgery as stents, catheters or tissue repair scaffolds. In applications where cell attachment and tissue integration is critical, most synthetic polymers may have limitations, see Sionkowska A. Prog Polym Sci. 2011; 36: 1254-1267. For example, polycaprolactone (PCL) is well-researched biomaterial with a long degradation time and robust mechanical properties that make it suitable for implanted structures such as scaffolds and stents. Collagen has excellent cell affinity, but lacks desirable mechanical properties.

### SUMMARY OF THE INVENTION

In view of the above, a problem of the present invention was to develop a device that can be utilized to fabricate hollow cylindrical medical devices, preferably including biomaterials that offer enhanced cell adhesion. This problem as well as others are solved by the devices, methods and hollow cylindrical constructs of the present invention described herein.

In one embodiment, the device includes a mandrel having a length along a central longitudinal axis, a spray device having a nozzle, the nozzle capable of applying a composition to one or more of an inner surface of the mandrel, an outer surface of the mandrel and another layer in contact with the mandrel; and a spinning fixture that a) provides rotation between the mandrel and the nozzle about the central longitudinal axis and b) provides movement along the central longitudinal axis such that the nozzle can be located at at least two different points along the central longitudinal axis of the mandrel to allow coating of one or more of the inner surface of the mandrel, the outer surface of the mandrel and the another layer in contact with the mandrel with the composition.

In a further embodiment a method for producing a hollow cylindrical structure is disclosed, comprising the steps of obtaining a device including a mandrel having a length along a central longitudinal axis, a spray device having a nozzle, the nozzle capable of applying a composition to one or more of an inner surface of the mandrel, an outer surface of the mandrel and another layer in contact with the mandrel; and a spinning fixture that a) provides rotation between the mandrel and the nozzle about the central longitudinal axis and b) provides movement along the central longitudinal axis such that the nozzle can be located at at least two different points along the central longitudinal axis of the mandrel to allow coating of one or more of the inner surface of the mandrel, the outer surface of the mandrel and the another layer in contact with the mandrel with the composition; spraying a first composition on the mandrel and/or another layer in contact in the mandrel; spraying a second composition on one or more of the mandrel and the first composition, wherein at least one of the first composition and the second composition comprise a biomaterial; and forming a hollow cylindrical structure comprising at least the first composition and the second composition.

In still a further embodiment, a hollow cylindrical structure is disclosed, comprising at least two different materials including at least crosslinked collagen, wherein the hollow cylindrical structure has a central longitudinal axis and a bore extending along the length of the central longitudinal axis.

As utilized herein, the term “hollow cylindrical structure” and derivatives thereof refer to any cylinder-like structure having an aperture therein extending from a first side to a second side. As height of the cylinder can vary, the structures may be considered to be tubes, rings, washers, toroids and the like. Hollow cylindrical structures are preferred in some embodiments.

### BRIEF DESCRIPTION OF THE DRAWINGS

The invention will be better understood and other features and advantages will become apparent by reading the detailed description of the invention, taken together with the drawings, wherein:

FIG. 1 illustrates one embodiment of a device for the construction and coating of a hollow cylindrical structure utilizing a spray device to spray at least one composition on a mandrel and/or another layer in contact with the mandrel, wherein the embodiment illustrated is particularly suitable for spraying a coating while inside a hollow section of the mandrel;

FIG. 2 illustrates one embodiment of a spray device including a nozzle adapted for spraying while disposed inside a hollow mandrel;

FIG. 3 illustrates one embodiment of a spray device having one nozzle for spraying on an outer surface of a mandrel and/or another layer on an outer surface of the mandrel;

FIG. 4 illustrates one embodiment of a spray device having multiple nozzles capable of spraying a composition on an outer surface of a mandrel and/or another layer on an outer surface of the mandrel;

FIG. 5 illustrates a top view of one embodiment of a spray device having two nozzles;

FIG. 6 illustrates a front view of FIG. 5;

FIG. 7 illustrates a further embodiment of a spray device having multiple nozzles for spraying multiple compositions and/or gases on a mandrel and/or workpiece;

FIG. 8 illustrates a close-up view of a nozzle arrangement for a spray device including three nozzles capable of spraying liquid compositions and an additional nozzle capable of directing gas which can serve to form a guide stream around the inner nozzle(s) providing for distribution of the liquid onto an outer surface of the mandrel and/or another layer on an outer surface of the mandrel;

FIG. 9 presents side view and top view images of one embodiment of a hollow cylindrical structure suitable for use as a tracheal stent, wherein the patterned apertures were added post-production;

FIG. 10 illustrates hollow cylindrical structures of various diameters and wall thicknesses fabricated by the devices and methods of the present invention;

FIG. 11 illustrates a cell proliferation on day 7 on (a) PCL sample and (b) collagen-PCL composite sample stained with actin and DAPI; and

FIG. 12 illustrates a) a further embodiment of a device for producing hollow cylindrical structures including a spray device having three fluid spaying nozzles, and b) illustrates a close-up view of the three nozzles capable of simultaneous spraying of up to three compositions at a time.

### DETAILED DESCRIPTION OF THE INVENTION

Devices are disclosed herein that are suitable for constructing hollow cylindrical structures and/or coating a pre-formed hollow cylindrical structure. The devices can fabricate hollow cylindrical structures having single or multiple layers, with each layer being formed of one or more different materials. Biomaterials are utilized in preferred embodiments. In some embodiments, multiple compositions can be sprayed on a mandrel or a layer of a hollow cylindrical structure simultaneously or sequentially utilizing the devices of the invention. The fabricated hollow cylindrical structures are particularly useful as medical devices in some embodiments. Certain biomaterials facilitate cell attachment and tissue integration upon placement within a patient.

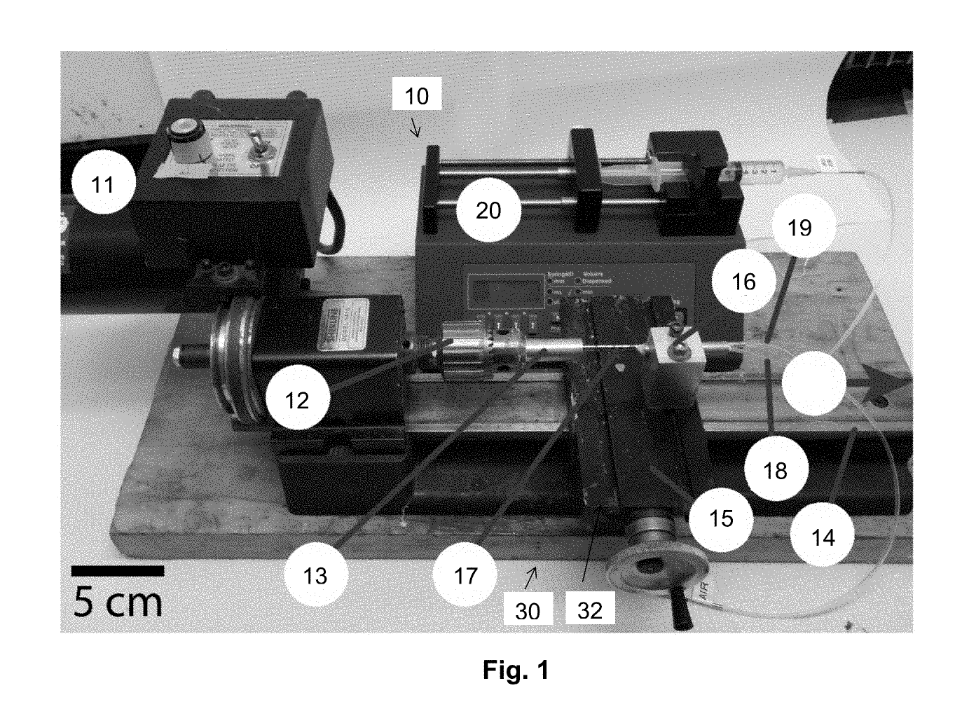

Referring now to the drawings, wherein like reference numbers denote like parts throughout the several views, FIG. 1 illustrates one embodiment of a device 10 for the construction and coating of a hollow cylindrical structure utilizing a spray device to spray at least one composition on a mandrel and/or at least one layer directly or indirectly in contact with the mandrel, wherein the embodiment illustrated is particularly suitable for coating the inside of a mandrel 13. Device 10 includes a motor assembly 11 comprising a motor and speed controller operatively connected to a rotatable chuck 12 to which a desired mandrel 13 is connected. Mandrel 13 has a longitudinal length with a central longitudinal axis. While the mandrel is shown generally as a substantially cylindrical rod, it is to be understood that a mandrel having other shapes and configurations can be utilized. For example, mandrels can have other shapes such as non-cylindrical cross-sections including, but not limited to, rectangles and hexagons. The speed of mandrel rotation will vary depending on the type of solution applied thereto. Speed of rotation also depends upon whether or not the coating is applied to the inside of the mandrel or outside of the mandrel as centrifugal forces can prevent the solution from staying on the mandrel when a coating is applied to the outside of a mandrel. However, centrifugal forces can be used to create a more dense structure at a relatively higher speed of rotation. For sake of clarity, it is to be understood that the mandrel can rotate clockwise or counterclockwise in relation to the longitudinal axis.

The mandrel illustrated in FIG. 1 has a hollow portion such that a part of spray device 17 can be placed therewithin in order to coat an inner surface of the mandrel and/or at least one layer directly or indirectly in contact with the mandrel with a composition.

The device 10 also includes a carriage assembly 30 including a track 14 to which a carriage 32 is operatively mounted. Carriage 32 is moveable along the central longitudinal axis of track 14. Spray device 17 is operatively mounted on carriage 32. Spray device 17 includes a spray holding post 16 which fixes the same to carriage 32. Various fluid lines that are able to convey one or more of a gas, such as air, and a liquid composition, for example connection lines 18 and 19, are operatively connected to spray device 17.

The embodiment also illustrates solution supply 20 connected to fluid line 19. Any pump can be used to provide a solution supply. Preferably, a syringe pump, a constant pressure pump, or a peristaltic pump will be used to accurately control the amount of solution supplied. Alternatively, the solution may be gravity fed into the nozzle from a reservoir, using the Venturi effect to draw fluid.

Connection line 18 is connected to a gas source, such as a compressor or air tank in one embodiment. Carriage 32 can be controlled manually or with a suitable drive such as a solenoid valve. The carriage 32 can be moved along the central longitudinal axis along the track using a leadscrew, drive belt, or rack and pinion mechanism with the motion being controlled manually or automatically via a motor and a computerized controller. The computerized controller controls the position of the motor to allow precise control of the speed of motion and thus thickness of each layer, the number of layers of materials sprayed and the distribution of material along the length of the mandrel. In one embodiment the drive system utilizes and open loop control with a stepper motor. A closed feedback loop may be incorporated to track the position of the carriage and change the speed of the motor in real time.

As can be taken from FIG. 1, mandrel 13 is rotated in relation to spray device 17. The spray device 17 can spray a composition on an inner surface of mandrel 13 or on an inner surface of a hollow cylindrical structure placed or formed inside the mandrel, if desired.

Distance between the central longitudinal axis of the mandrel 13 and a nozzle of a spray device can be adjusted by utilizing saddle 15.

The saddle moves on rails placed on the carriage assembly and is driven by a manual leadscrew in one configuration. A drive system as described above can be used to control the position through the controller in another embodiment.

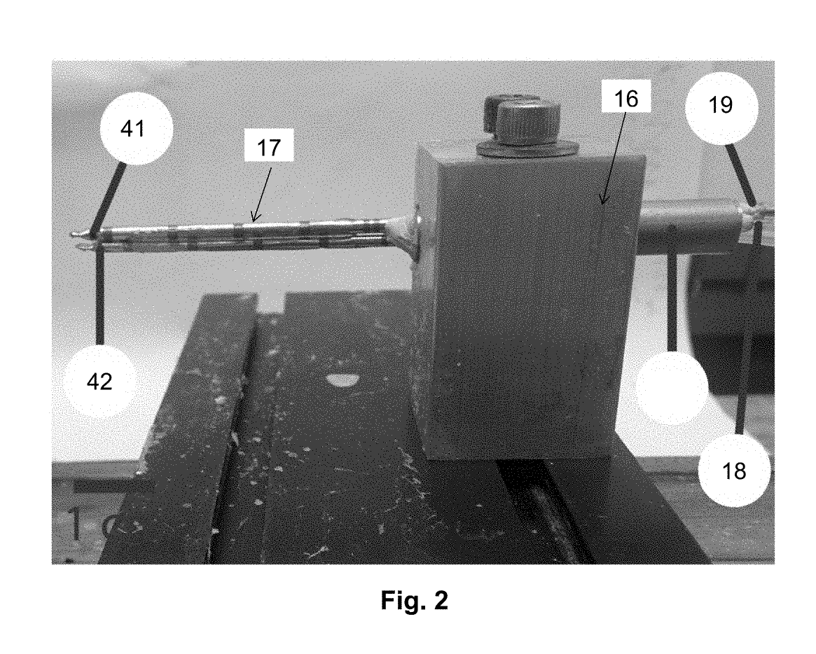

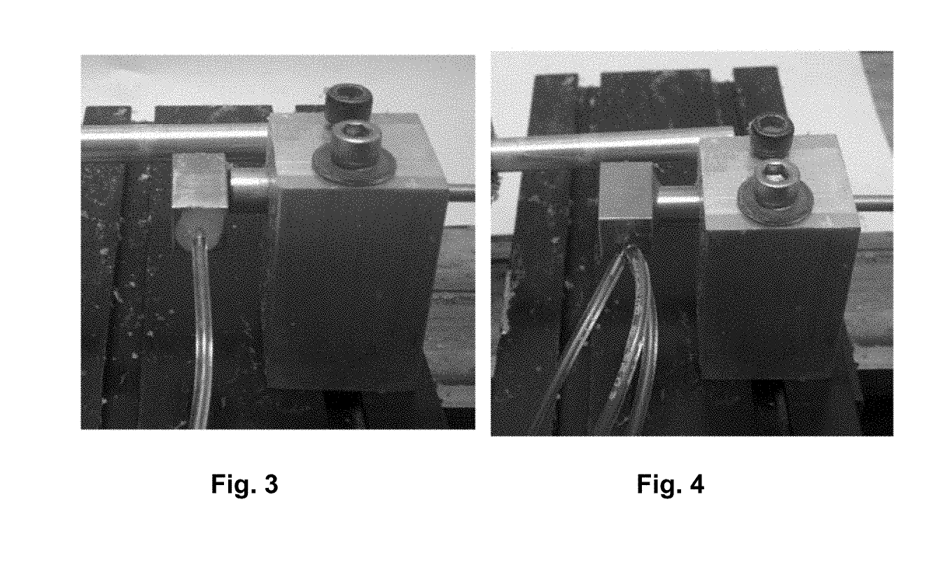

It is further important to note that while the spray holding post 16 is shown attached to carriage 32 in a particular position, the location may be changed along the length of the carriage as well as the angle relative to the central longitudinal axis of the mandrel. Spray holding post 16 is compatible with a large number of spray devices, see for example FIGS. 2-4 of the application.

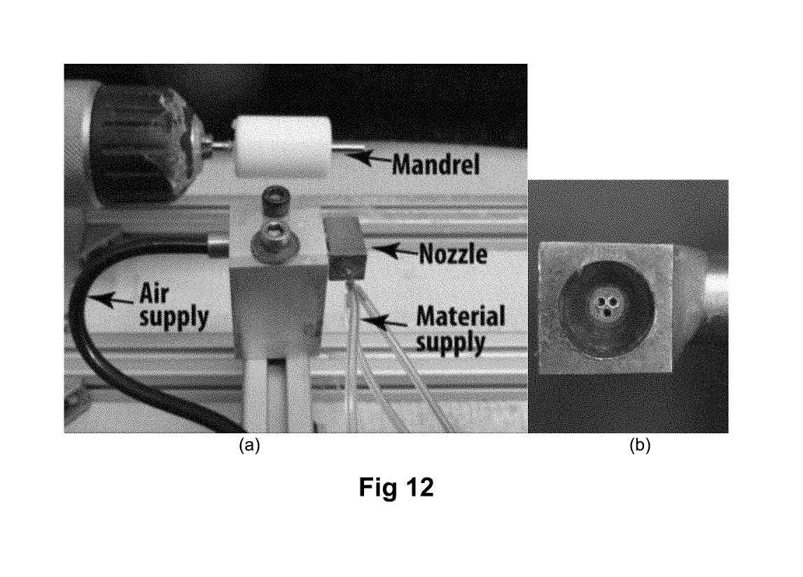

It should be further clear, that due to the versatility of the device, spray device 17 can have nozzles positioned to spray an outer surface of mandrel 13, see for example FIG. 12.

The solution supply 20 feeds the spray device with a composition, preferably a liquid to be sprayed. A compressor or air tank can provide pressurized air to propel the solution towards the mandrel or other workpiece.

The compositions that can be sprayed by the device 10 may vary depending upon the form of the hollow cylindrical structure desired to be constructed. In some embodiments, the liquid comprises a solution of desired material in a volatile solvent. Hollow cylindrical structure-forming materials include, but are not limited to, polycaprolactone, polylactic-co-glycolic acid) (PGLA) and collagen. Any composition able to dissolve the composition desired to form the hollow cylindrical structure can be utilized as a solvent. Suitable solvents include by are not limited to, chloroform, acetic acid, and water, for example. Concentration of the hollow cylindrical structure-forming components can vary. In one embodiment 4%-10% by weight of polycaprolactone is utilized in chloroform, optionally with up to 200 mf/mL of mometasone furoate. Additional compositions include 1-10 mg/ml of collagen in an aqueous solution or in an acetic acid solution. Solutions of PGLA, such as at a concentration of 4%, in chloroform are also suitable.

As the mandrel or a layer of hollow cylindrical structure is sprayed with the composition, the solvent evaporates, leaving the solute, i.e. the construction material on the surface on which it has been sprayed. Depending upon a thickness desired, the hollow cylindrical structure can be obtained using one or more layers of sprayed compositions. The solution supply and a suitable controller allow precise control over the amounts of material to be dispensed.

The devices and methods of the present invention allow a wide variety of soluble materials to be utilized in order to fabricate hollow cylindrical structures, such as rigid-walled structures having desired cross-sections, substantially round cross-sections in preferred embodiments. Various different materials can be utilized simultaneously or sequentially to form a hollow cylindrical structure having a particular construction and desired properties.

In some embodiments a pre-formed hollow cylindrical structure is placed inside a mandrel and thereafter spray coated utilizing a device of the present invention. The pre-formed hollow cylindrical structure can be a solid structure or have pores. In some embodiments, the pre-formed structure can be formed by knitting a suitable composition. In other embodiments, a hollow cylindrical structure can be formed by spraying an inner surface of mandrel 13. To this end, a spray device 17 capable of spraying while inside a section of a hollow mandrel is shown in FIG. 2. Therein, spray device 17 is shown including a nozzle 1 capable of spraying a liquid composition and a second nozzle 42 capable of spraying a gas such as air. Spray device 17 is designed to fit inside the mandrel and/or hollow cylindrical structure and deliver a spray coating to an inside surface thereof, generally from a short distance. The nozzles, 41 and 42, are preferably disposed perpendicular or substantially perpendicular to the central longitudinal axis of rotation of the mandrel.

A liquid composition is supplied from a solution supply, such as shown in FIG. 1. The feed rate of solution supply and gas pressure can be varied depending upon the type of composition to be applied and the thicknesses desired.

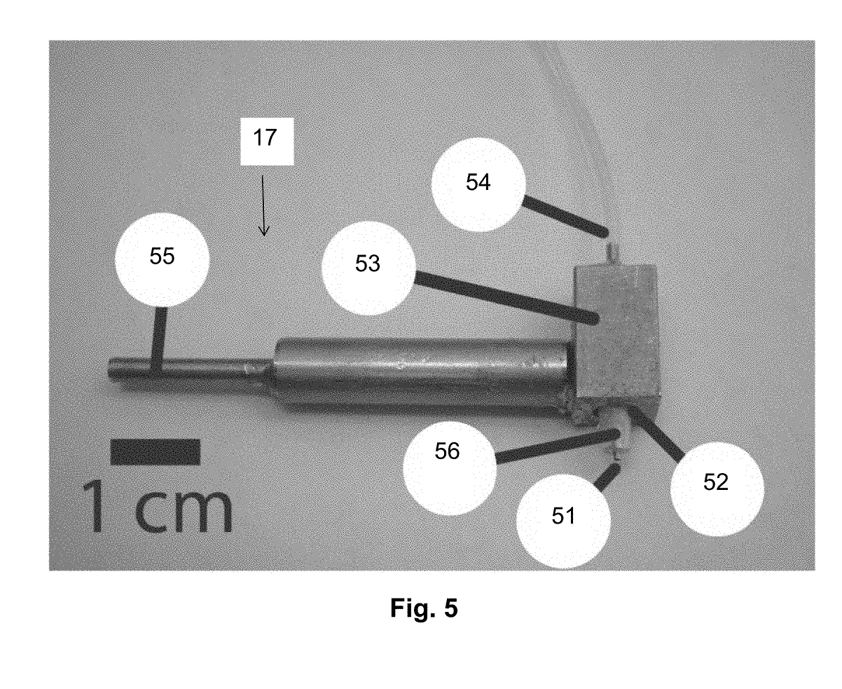

FIG. 5 illustrates a single nozzle spray device for spraying on a mandrel or workpiece. As compared to spray device 16, such as shown in FIG. 4 that is adapted to spray an inner surface of a mandrel or workpiece, the spray device 16 shown in FIG. 5 can spray from a relatively longer distance and utilize relatively higher gas pressure. A liquid is supplied by a solution supply, not shown, to liquid supply port 54 which is expelled through nozzle 51. When desired, the gas can be supplied to gas supply port 55 and emitted through gas nozzle 52. Body 53 houses nozzles 51 and 52 which are concentric with each other, with the liquid-spraying nozzle 51 located inside gas nozzle 52. Cone 56 guides the gas stream from gas nozzle 2 around liquid nozzle 51, providing for a more even distribution of the liquid.

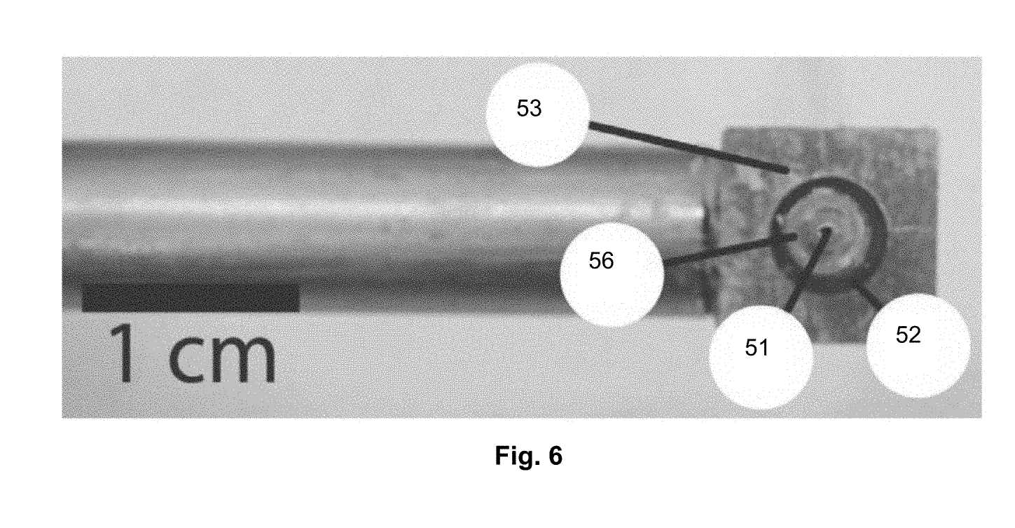

FIG. 6 shows a front view of the embodiment shown in FIG. 5 particularly illustrating the structural arrangement between nozzles 51 and 52.

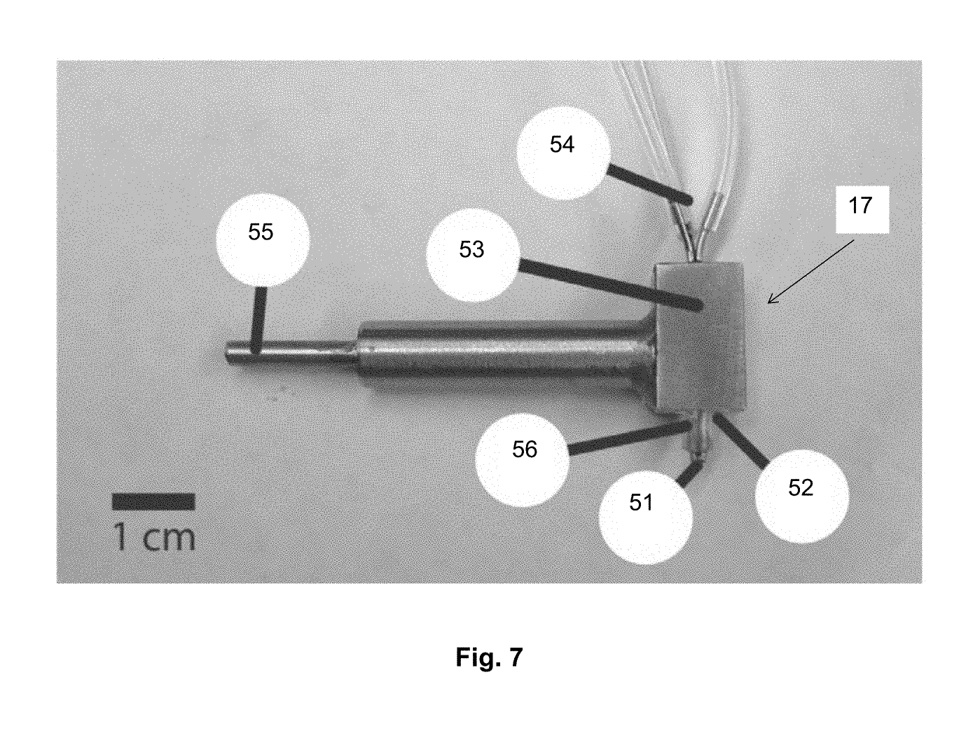

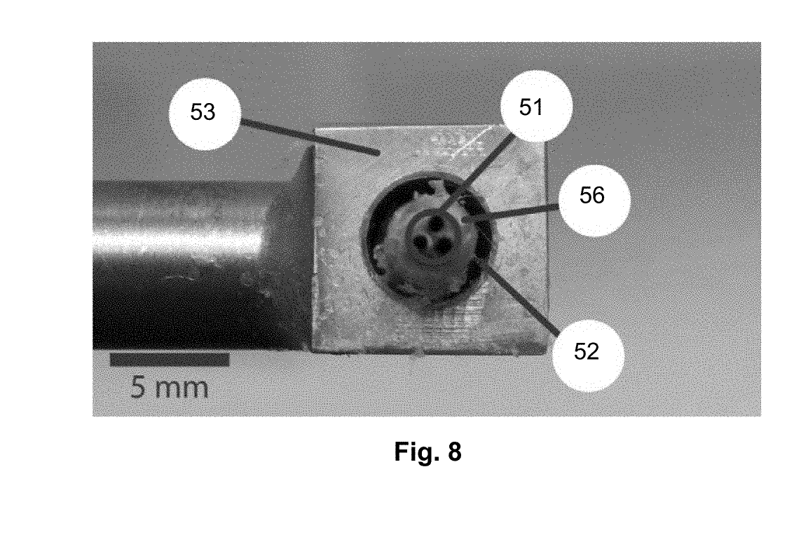

FIG. 7 illustrates a further embodiment of a multi-nozzle spray device 17 for spraying on a mandrel or workpiece. The embodiment is similar to the arrangement shown in FIG. 5 but has a plurality of liquid-spraying nozzles 51, in particular three nozzles in this embodiment. The front view is illustrated in FIG. 8. The three nozzles 51 can be used together or independently. This device allows for combining materials that cannot be easily mixed using a common solvent, using different materials for each layer of the sprayed structure, and complete automation of producing a multi-material hollow cylindrical structure.

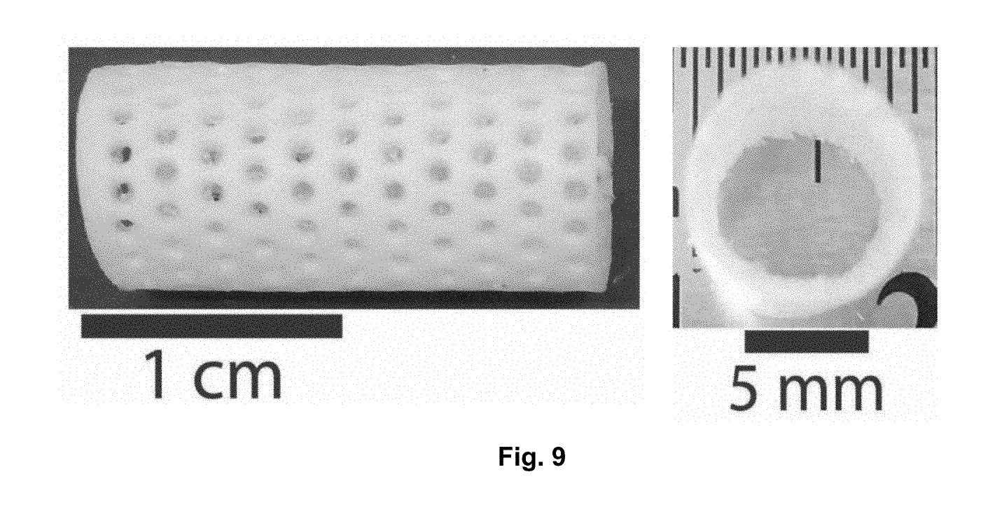

FIG. 9 illustrates top and side views of a hollow cylindrical structure formed with the present invention. The hollow cylindrical structure can be utilized as a tracheal stent. The pattern holes were added post-production Using a 4 axis CNC milling machine. The device illustrated in FIG. 9 is a bio-absorbable stent formed from polycaprolactone. The device was utilized to spray coat an inner surface of the structure with collagen. In a preferred embodiment a post-processing crosslinking step is performed in order to crosslink the collagen. Genipin is utilized in one embodiment. In order embodiments EDC-NHS is utilized.

Hollow cylindrical structures can be formed with generally any diameter and wall thickness desired by the fabricator. That said, minimum diameters range from less than 1 mm to at least 3 cm, although larger and smaller diameters can be produced. Length of the hollow cylindrical structures range generally from about 1 mm to about 60 cm. Longer structures can be produced, if desired. The thicknesses range generally from about 10 microns to about 10 mm as measured in a radial direction. If more than one layer of material is utilized, the thickness for each layer can vary such that the total thickness of the hollow cylindrical structure is within the thickness range indicated.

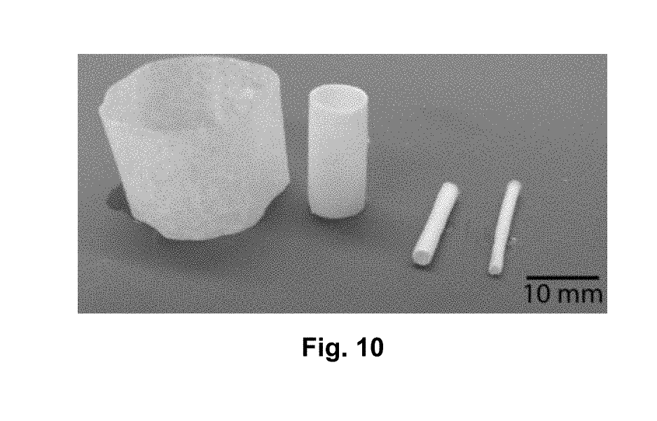

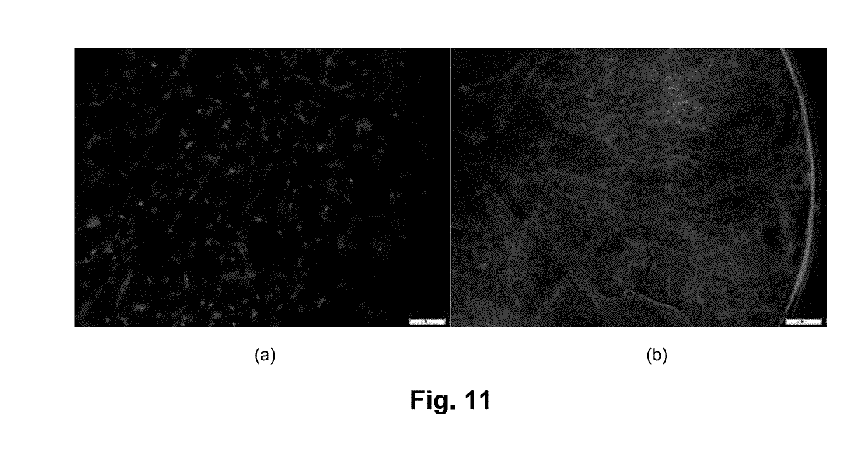

FIG. 10 illustrates additional hollow cylindrical structures of various diameters and wall thicknesses fabricated using the devices of the present invention.

FIG. 12 illustrates a further embodiment of a device of the present invention capable of spraying up to three different materials on a mandrel or workpiece. The presence of three nozzles allows capability of simultaneously spraying three materials at a time or sequentially spraying three materials. The device is essentially a two dimensional computer controlled fabrication tool where one axis is a rotating mandrel and the other axis is a translation spray device having nozzles for spray-coating layers of material on the spinning mandrel or a prior construct connected to a mandrel. The method allows for co-spraying materials that are otherwise immiscible, for example collagen and synthetic polymers.

### EXAMPLES

Polymer Solutions: Type I bovine collagen was dissolved in 50% v/v water-ethanol at 10 mg/ml and sprayed to form a pure collagen lumen. 4% PCL (80,000 g/mol) dissolved in chloroform was sprayed to form the outermost layer. A transition layer was applied after the collagen layer by co-spraying the two solutions to ensure adhesion between the layers. The construct was treated with genipin to crosslink the collagen network.

Mechanical tests: Rectangular strips were cut from fabricated tubes and tensile tests were performed to measure the strength and modulus of the material. Cell assays: Human mesenchymal stem cells (MSCs) were seeded on PCL and PCL-collagen composite samples to determine the benefits of the added collagen. A live/dead assay was performed 24 hours after seeding. Cell proliferation was quantified on day 1 and day 7.

Results: Hollow cylindrical constructs of various sizes were fabricated as shown in FIG. 10. The elastic modulus of the fabricated PCL samples was 91.8±16.6 MPa, and the yield stress was 5.5±1.2 MPa. The variation is largely due to random orientation and porosity of the construct, as confirmed by SEM. Live/dead assay showed 100% of the cells on the PCL-collagen composite sample were alive 24 hours after seeding, in contrast to the 64±18% of the cells on PCL samples. Day 7 proliferation showed 1112±97 cells/mm2 on the PCL-collagen sample but only 95±9 cells/mm2 on the PCL sample 11. PCL-collagen composite was shown more habitable for cells than PCL (p<0.05).

Conclusions: The device and method enabled the addition of collagen to PCL in constructing hollow cylindrical forms in a computer-controlled fashion. The method enables co-spraying, sequential spraying, all in a spatially controlled format. importantly, incorporation of collagen to the composite favored cell growth and proliferation while taking advantage of the mechanical robustness provided by PCL alone.

For the avoidance of doubt, the devices of the present invention encompass all possible combinations of the components, including various ranges of said components, disclosed herein. It is further noted that the term “comprising” does not exclude the presence of other elements. However, it is to also be understood that a description of a product or composition comprising certain components also discloses a product consisting of said components.

In accordance with the patent statutes, the best mode and preferred embodiment have been set forth; the scope of the invention is not limited thereto, but rather by the scope of the attached claims.

What is claimed is:

1. A device for producing a hollow cylindrical structure, comprising:
a mandrel having a length along a central longitudinal axis;
a spray device having a nozzle, the nozzle capable of applying a composition to one or more of an inner surface of the mandrel, an outer surface of the mandrel and a layer in direct or indirect contact with the mandrel; and
a spinning fixture that a) provides rotation between the mandrel and the nozzle about the central longitudinal axis and b) provides movement along the central longitudinal axis such that the nozzle can be located at at least two different points along the central longitudinal axis of the mandrel to allow coating of one or more of the inner surface of the mandrel, the outer surface and the layer in direct or indirect contact with the mandrel with the composition.

2. The device according to claim 1, wherein the spinning fixture comprises a motor and a chuck operatively connected to the motor, wherein the mandrel is operatively connected to the chuck which rotates the mandrel about the central longitudinal axis.

3. The device according to claim 2, wherein the spinning fixture includes a carriage assembly including a track, and a carriage operatively mounted on the track and moveable along the central longitudinal axis, and wherein the spray device is mounted on the carriage.

4. The device according to claim 3, wherein the mandrel has at least one hollow section, wherein the carriage is able to move the nozzle so that it can be located at least at two different points along the central longitudinal axis inside the at least one hollow section.

5. The device according to claim 4, wherein the nozzle has an outlet that is substantially perpendicular to the central longitudinal axis.

6. The device according to claim 3, wherein the carriage is moveable such that the nozzle can be located at least two different points along the central longitudinal axis to allow coating of the outer surface of the mandrel.

7. The device according to claim 6, wherein the nozzle comprises a pair of concentric nozzles wherein an inner nozzle is adapted to spray a liquid therefrom and an outer nozzle is adapted to spray a gas therefrom, wherein the gas guides an air stream around the inner nozzle providing for distribution of the liquid onto the outer surface of the mandrel.

8. The device according to claim 7, wherein a plurality of inner nozzles are provided, each operated independently and able to spray a liquid coating simultaneously or at different times as compared to at least another nozzle.

9. The device according to claim 3, wherein a controller controls one or more of the following features: speed of rotation of the mandrel about the central longitudinal axis, movement of the carriage along the central longitudinal axis, functions of the nozzle and distribution of material along a length of the mandrel.

10. A method for producing a hollow cylindrical structure, comprising the steps of:
obtaining the device according to claim 1;
spraying a first composition on one or more of an inner surface of the mandrel, an outer surface of the mandrel and the layer in direct or indirect contact with the mandrel with the composition;
spraying a second composition on one or more of the mandrel and the first composition, wherein at least one of the first composition and the second composition comprise a biomaterial; and
forming a hollow cylindrical structure comprising at least the first composition and the second composition.

11. The method according to claim 10, wherein the first composition is applied to the mandrel prior to the second composition being applied to the first composition.

12. The method according to claim 10, wherein the first composition and the second composition are sprayed on the mandrel at the same time.

13. The method according to claim 10, wherein the biomaterial comprise collagen.

14. The method according to claim 13, further including the step of crosslinking the collagen after forming the hollow cylindrical structure.

15. The method according to claim 14, wherein the collagen is crosslinked with genipin or EDC-NHS.

16. A hollow cylindrical structure, comprising:
at least two different materials including at least crosslinked collagen, wherein the hollow cylindrical structure has a central longitudinal axis and a bore extending along the length of the central longitudinal axis.

17. The hollow cylindrical structure according to claim 16, wherein the hollow cylindrical structure includes at least two layers in relation to an axis radial to the central longitudinal axis, and wherein an outer layer comprises the crosslinked collagen.

18. The hollow cylindrical structure according to claim 17, wherein an inner layer comprises polycaprolactone.
